(19) United States
(12) Patent Application Publication
(10) Pub. No.: US 2024/0335447 A1
(43) Pub. Date: Oct. 10, 2024

(54) WEE1 INHIBITOR FOR CANCER

(71) Applicant: Recurium IP Holdings, LLC, San Diego, CA (US)

(72) Inventors: Fernando Donate, Santa Monica, CA (US); Hooman Izadi, San Diego, CA (US); Petrus Rudolf de Jong, San Diego, CA (US); Ahmed Abdi Samatar, West Windsor, NJ (US); Kevin Duane Bunker, Escondido, CA (US); Peter Qinhua Huang, San Diego, CA (US)

(21) Appl. No.: 18/743,544

(22) Filed: Jun. 14, 2024

Related U.S. Application Data
(63) Continuation of application No. PCT/US2022/081596, filed on Dec. 14, 2022.
(60) Provisional application No. 63/265,438, filed on Dec. 15, 2021.

(51) Int. Cl. A61K 31/519; A61P 35/00

(52) U.S. Cl. CPC A61K 31/519; A61P 35/00

(57) ABSTRACT

Disclosed herein is a WEE1 compound, or a pharmaceutically acceptable salt thereof, alone or in combination with a KRAS inhibitor, or a pharmaceutically acceptable salt thereof, for treating a disease or condition, such as a colorectal, pancreatic and/or non-small cell lung cancer.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6, including U.S. Provisional Application No. 63/265,438, filed Dec. 15, 2021, which is incorporated by reference in its entirety. The present application is a continuation of PCT Application No. PCT/US2022/081596, filed Dec. 14, 2022, which claims priority to U.S. Provisional Application No. 63/265,438, filed Dec. 15, 2021, each of which is hereby incorporated by reference in their entireties.

### FIELD

The present application relates to the fields of chemistry, biochemistry and medicine. More particularly, disclosed herein are combination therapies, and methods of treating diseases and/or conditions with a combination therapy descried herein.

### DESCRIPTION

Cancers are a family of diseases that involve abnormal cell growth with the potential to invade or spread to other parts of the body. Cancer treatments today include surgery, hormone therapy, radiation, chemotherapy, immunotherapy, targeted therapy and combinations thereof. Survival rates vary by cancer type and by the stage at which the cancer is diagnosed. In 2021, roughly 1.9 million people will be diagnosed with cancer, and an estimated 600,000 people will die of cancer in the United States. Thus, there still exists a need for effective cancer treatments. Colorectal cancer is one of the most common cancers in both men and women worldwide.

### SUMMARY

Some embodiments described herein relate to the use of an effective amount of Compound (A) and/or Compound (B), or a pharmaceutically acceptable salt of any of the foregoing, for treating a cancer selected from colorectal cancer, a pancreatic cancer and a non-small cell lung cancer (NSCLC) in a subject having a mutation selected from a TP53 and a KRAS mutation. Other embodiments described herein relate to the use of an effective amount of Compound (A) and/or Compound (B), or a pharmaceutically acceptable salt of any of the foregoing, in the manufacture of a medicament for treating a cancer selected from colorectal cancer, a pancreatic cancer and NSCLC in a subject having a mutation selected from a TP53 and a KRAS mutation. Still other embodiments described herein relate to a method of treating a cancer that can include administering a combination of compounds, wherein the combination includes an effective amount of Compound (A) and/or Compound (B), or a pharmaceutically acceptable salt of any of the foregoing; and wherein the cancer can be selected from colorectal cancer, a pancreatic cancer and NSCLC; in a subject having a mutation selected from a TP53 and a KRAS mutation.

Some embodiments described herein relate to a combination of compounds that can include an effective amount of Compound (A) and/or Compound (B), or a pharmaceutically acceptable salt of any of the foregoing, and an effective amount of a KRAS inhibitor, or a pharmaceutically acceptable salt thereof.

Some embodiments described herein relate to the use of a combination of compounds for treating a cancer selected from colorectal cancer, a pancreatic cancer and NSCLC in a subject having a mutation selected from a TP53 and a KRAS mutation, wherein the combination includes an effective amount of Compound (A) and/or Compound (B), or a pharmaceutically acceptable salt of any of the foregoing, and an effective amount of a KRAS inhibitor, or a pharmaceutically acceptable salt thereof. Other embodiments described herein relate to the use of a combination of compounds in the manufacture of a medicament for treating a cancer selected from colorectal cancer, a pancreatic cancer and NSCLC in a subject having a mutation selected from a TP53 and a KRAS mutation, wherein the combination includes an effective amount of Compound (A) and/or Compound (B), or a pharmaceutically acceptable salt of any of the foregoing, and an effective amount of a KRAS inhibitor, or a pharmaceutically acceptable salt thereof. Still other embodiments described herein relate to a method of treating a cancer that can include administering a combination of compounds, wherein the combination includes an effective amount of Compound (A) and/or Compound (B), or a pharmaceutically acceptable salt of any of the foregoing, and an effective amount of a KRAS inhibitor, or a pharmaceutically acceptable salt thereof; and wherein the cancer can be selected from colorectal cancer, a pancreatic cancer and NSCLC; in a subject having a mutation selected from a TP53 and a KRAS mutation.

### DRAWINGS

FIG. 1 provides examples of KRAS inhibitors.

FIG. 2 shows the effect of using Compound (A), or a pharmaceutically acceptable salt thereof, and a KRAS inhibitor alone or in combination on tumor volume in a H23 non-small cell lung model.

FIG. 3 shows the effect of using Compound (A), or a pharmaceutically acceptable salt thereof, and a KRAS inhibitor alone or in combination on tumor volume in a MiaPaca-2 pancreatic model.

FIG. 4 shows the effect of using Compound (A), or a pharmaceutically acceptable salt thereof, and a KRAS inhibitor alone or in combination on tumor volume in a H358 non-small cell lung model.

FIG. 5 shows the effect of using Compound (A), or a pharmaceutically acceptable salt thereof, and a KRAS inhibitor alone or in combination on tumor volume in a SW837 CRC adenocarcinoma model.

FIG. 6 shows the effect of using Compound (A), or a pharmaceutically acceptable salt thereof, and a KRAS inhibitor alone or in combination on tumor volume in an SW837 CRC adenocarcinoma model.

FIG. 7 shows the effect of using Compound (A), or a pharmaceutically acceptable salt thereof, in a colorectal cancer LoVo xenograft model.

FIG. 8 shows the effect of using Compound (A), or a pharmaceutically acceptable salt thereof, in a colorectal cancer SW1116 xenograft model.

FIG. 9 illustrates representative assay data obtained for Compound (A), or a pharmaceutically acceptable salt thereof, and a KRAS inhibitor (Sotorasib) in a MiaPaca-2 (pancreatic cancer) cell line. The results show that surprisingly, the combination of Compound (A), or a pharmaceutically acceptable salt thereof, and a KRAS inhibitor resulted in synergistic activity.

FIG. 10 illustrates representative assay data obtained for Compound (A), or a pharmaceutically acceptable salt thereof, and a KRAS inhibitor (MRTX849) in a MiaPaca-2 (pancreatic cancer) cell line. The results show that surprisingly, the combination of Compound (A), or a pharmaceutically acceptable salt thereof, and another KRAS inhibitor resulted in synergistic activity.

FIG. 11 illustrates representative assay data obtained for Compound (A), or a pharmaceutically acceptable salt thereof, and a KRAS inhibitor (Sotorasib) in a SW1463 (colorectal adenocarcinoma) cell line. The results show that surprisingly, the combination of Compound (A), or a pharmaceutically acceptable salt thereof, and a KRAS inhibitor resulted in synergistic activity in a second cell line.

### DETAILED DESCRIPTION

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there is a plurality of definitions for a term herein, those in this section prevail unless stated otherwise.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), a sulfuric acid, a nitric acid and a phosphoric acid (such as 2,3-dihydroxypropyl dihydrogen phosphate). Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, trifluoroacetic, benzoic, salicylic, 2-oxopentanedioic or naphthalenesulfonic acid. Pharmaceutical salts can also be obtained by reacting a compound with a base to form a salt such as an ammonium salt, an alkali metal salt, such as a sodium, a potassium or a lithium salt, an alkaline earth metal salt, such as a calcium or a magnesium salt, a salt of a carbonate, a salt of a bicarbonate, a salt of organic bases such as dicyclohexylamine, N-methyl-D-glucamine, tris(hydroxymethyl)methylamine, C₁-C₇ alkylamine, cyclohexylamine, triethanolamine, ethylenediamine and salts with amino acids such as arginine and lysine. Those skilled in the art understand that when a salt is formed by protonation of a nitrogen-based group (for example, NH₂), the nitrogen-based group can be associated with a positive charge (for example, NH₂ can become NH₃⁺) and the positive charge can be balanced by a negatively charged counterion (such as Cl⁻).

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomeric ally pure, enantiomerically enriched, racemic mixture, diastereomerically pure, diastereomerically enriched or a stereoisomeric mixture. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates and hydrates. In some embodiments, the compounds described herein exist in solvated forms with pharmaceutically acceptable solvents such as water, ethanol or the like. In other embodiments, the compounds described herein exist in unsolvated form. Solvates contain either stoichiometric or non-stoichiometric amounts of a solvent and may be formed during the process of crystallization with pharmaceutically acceptable solvents such as water, ethanol or the like. Hydrates are formed when the solvent is water or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a compound, composition or device, the term “comprising” means that the compound, composition or device includes at least the recited features or components but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Compounds

Some embodiments described herein relate to the use of an effective amount of Compound (A) and/or Compound (B), or a pharmaceutically acceptable salt of any of the foregoing, for treating a colorectal cancer in a subject having a mutation selected from a TP53 and a KRAS mutation.

The human TP53 gene is located on chromosome 17p, and consists of 11 exons and 10 introns. The wild type p53 protein consists of 393 amino acid residues. Several p53 mutations have been identified in colorectal cancer. Examples of p53 mutations include those described in Li et al., World J Gastroenterol (2015) 21(1):84-93 and Bouaoun et al., Hum Mutat. (2016) 37(9):865-876. KRAS mutations are believed to be one of the most frequent and prevalent in cancers, including colorectal cancer, pancreatic cancer and NSCLC. (See Maitra R, (2021). Therapeutic Approach to KRAS Mutated Colorectal Cancer. Cancer Therapy, MedDocs Publishers. Vol. 4, Chapter 1, pp. 1-5 and J. Luo, Semin Oncol. (2021) 48(1):10-18). KRAS mutations occur most commonly in codon 12, 13, 59 or 61 (including KRAS G12A, G12C, G12D, G12F, G12L, G12R, G12S, G12V, G12Y, G13A, G13C, G13D, G13R, G13S, G13V, A59T, Q61E, Q61H, Q61K, Q61L, Q61P, and Q61R), and less common other KRAS codons including codon 117 or 146 (including KRAS K117N, A146P, A146T or A146V). (See Moore et al., Nat. Rev. Drug Discov. (2020) 19(8):533-552.

Some embodiments disclosed herein relate to the use of a combination of compounds for treating a cancer selected from a colorectal cancer, a pancreatic cancer and NSCLC in a subject having a mutation selected from a TP53 and a KRAS mutation, wherein the combination can include an effective amount of Compound (A) and/or Compound (B), or a pharmaceutically acceptable salt of any of the foregoing, and an effective amount of a KRAS inhibitor, or a pharmaceutically acceptable salt thereof.

Some embodiments described herein relate to the use of an effective amount of AZD-1775 (hereinafter “Compound (B)”) and a KRAS inhibitor (such as those described herein), or pharmaceutically acceptable salts of any of the foregoing, for treating a cancer selected from a colorectal cancer, a pancreatic cancer and NSCLC in a subject having a mutation selected from a TP53 and a KRAS mutation.

Compound (A), including pharmaceutically acceptable salts thereof, can be

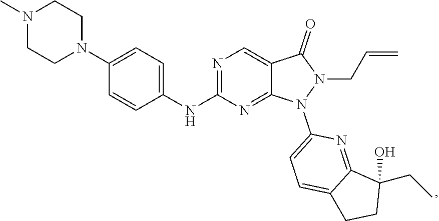 [SMILES: C=CCn1c(=O)c2cnc(Nc3ccc(N4CCN(C)CC4)cc3)nc2n1-c1ccc2c(n1)[C@@](O)(CC)CC2]

including pharmaceutically acceptable salts thereof. Examples of KRAS inhibitors include the following: sotorasib, adagrasib, JDQ443, MRTX-1257, MRTX1133, ARS-1620, ARS-853, ARS-107, BAY-293, BI-3406, BI-2852, BMS-214662, MRTX849, MRTX849-VHL (LC2), PROTAC K-Ras Degrader-1 (Compound 518, CAS No. 2378258-52-5), Lonafarnib (SCH66336), RMC-0331, GDC-6036, LY3537982, D-1553, ARS-3248 (JNJ74699157), BI-1701963 and AU-8653 (AU-BEI-8653).

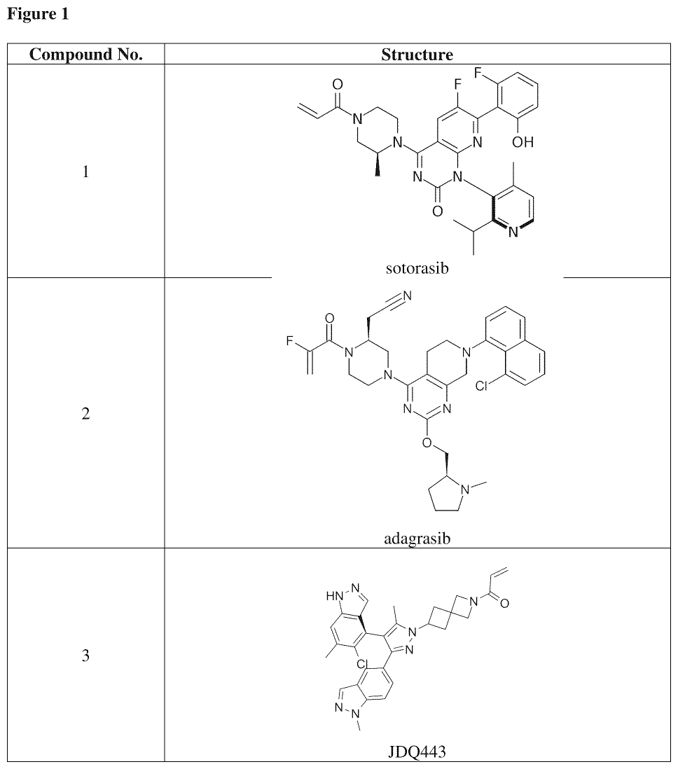

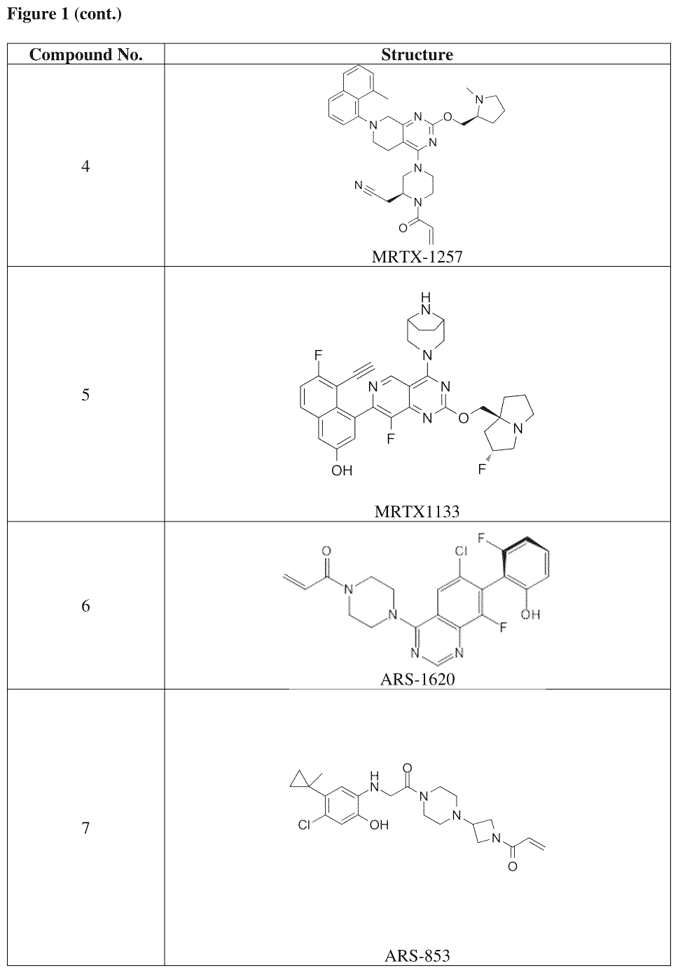

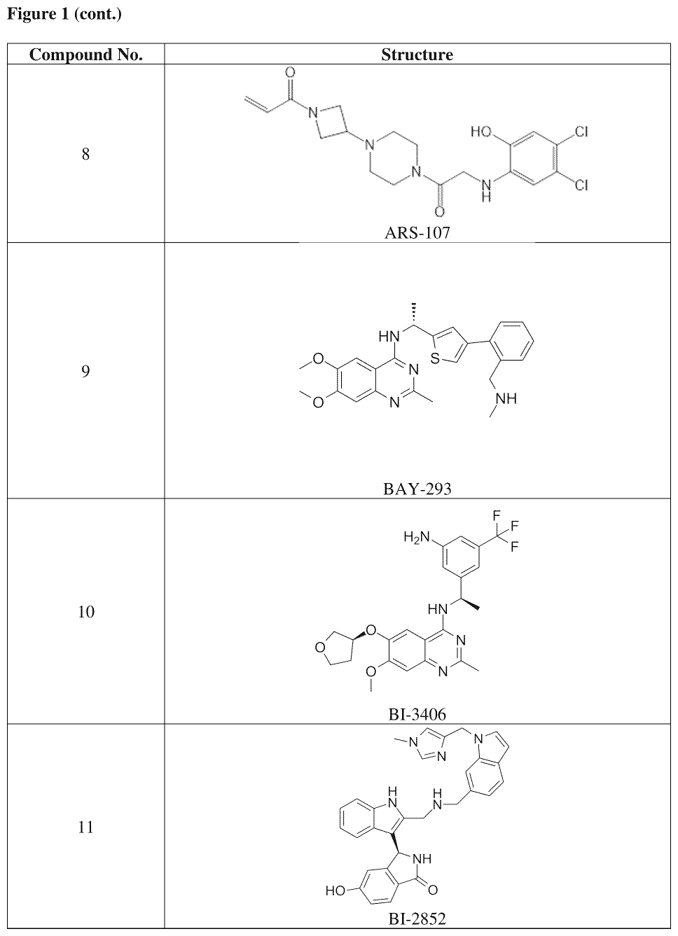

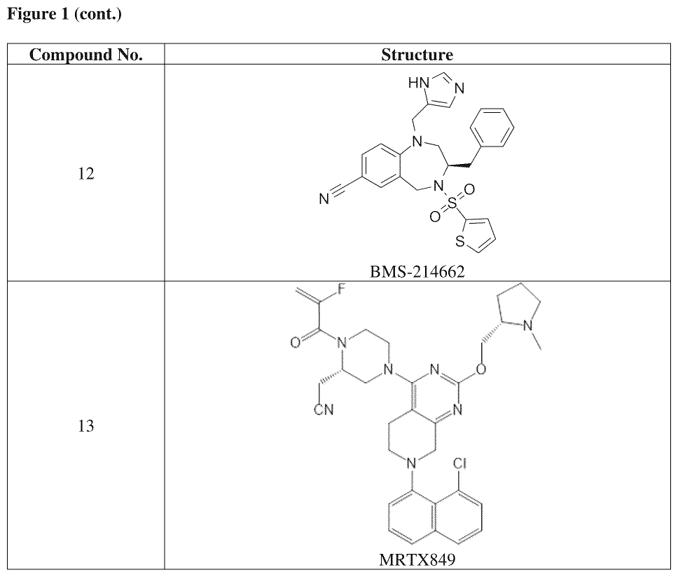

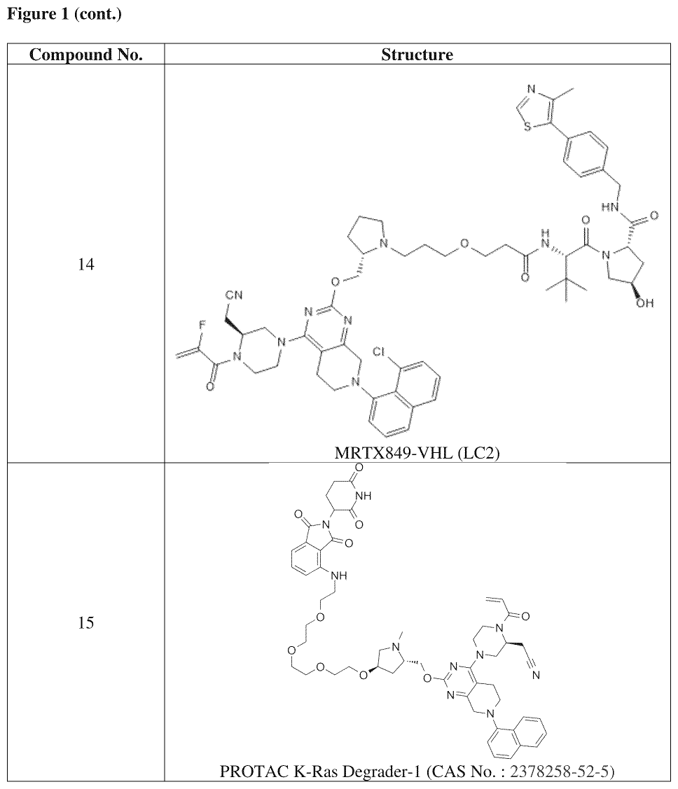

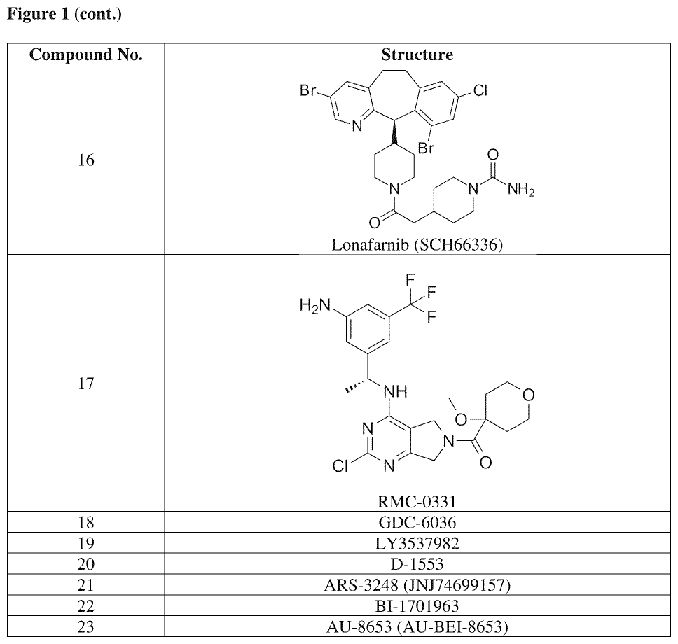

Embodiments of combinations of Compound (A) and a KRAS inhibitor, including pharmaceutically acceptable salts of any of the foregoing, and combinations of Compound (B) and a KRAS inhibitor, including pharmaceutically acceptable salts of any of the foregoing, are provided in Table 1. In Table 1, “A” indicates Compound (A) (including pharmaceutically acceptable salts thereof), “B” indicates Compound (B) (including pharmaceutically acceptable salts thereof) and the numbers 1-23 represent a compound as provided in FIG. 1, including pharmaceutically acceptable salts thereof. For example, in Table 1, a combination represented by 1:A corresponds to a combination of sotorasib and

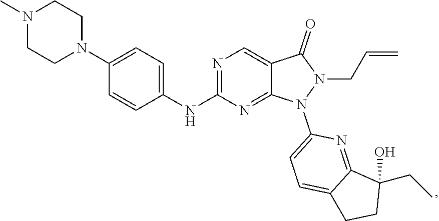 [SMILES: C=CCn1c(=O)c2cnc(Nc3ccc(N4CCN(C)CC4)cc3)nc2n1-c1ccc2c(n1)[C@@](O)(CC)CC2]

including pharmaceutically acceptable salts of any of the foregoing.

```
TABLE 1
Cmpd:Cmpd
  1:A
  2:A
  3:A
  4:A
  5:A
  6:A
  7:A
  8:A
  9:A
10:A
11:A
12:A
13:A
14:A
15:A
16:A
17:A
18:A
19:A
20:A
21:A
22:A
23:A
  1:B
  2:B
  3:B
  4:B
  5:B
  6:B
  7:B
  8:B
  9:B
10:B
11:B
12:B
13:B
14:B
15:B
16:B
17:B
18:B
19:B
20:B
21:B
22:B
23:B
```

When the treatment is a combination of compounds, the order of administration of compounds in a combination described herein can vary. In some embodiments, Compound (A) and/or Compound (B), including pharmaceutically acceptable salts of any of the foregoing, can be administered prior to all KRAS inhibitors, or a pharmaceutically acceptable salt of any of the foregoing. In other embodiments, Compound (A) and/or Compound (B), including pharmaceutically acceptable salts of any of the foregoing, can be administered prior to at least one a KRAS inhibitor, or a pharmaceutically acceptable salt thereof. In still other embodiments, Compound (A) and/or Compound (B), including pharmaceutically acceptable salts of any of the foregoing, can be administered concomitantly with a KRAS inhibitor, or a pharmaceutically acceptable salt thereof. In yet still other embodiments, Compound (A) and/or Compound (B), including pharmaceutically acceptable salts of any of the foregoing, can be administered subsequent to the administration of at least one KRAS inhibitor, or a pharmaceutically acceptable salt thereof. In some embodiments, Compound (A) and/or Compound (B), including pharmaceutically acceptable salts of any of the foregoing, can be administered subsequent to the administration of all KRAS inhibitors, or a pharmaceutically acceptable salt of any of the foregoing.

There may be several advantages for using a combination of compounds described herein. For example, combining compounds that attack multiple pathways at the same time, can be more effective in treating a cancer, such as those described herein, compared to when the compounds of combination are used as monotherapy.

In some embodiments, a compound described herein as mono-therapy and/or a combination as described herein of Compound (A) and/or Compound (B), including pharmaceutically acceptable salts of any of the foregoing, and a KRAS inhibitor, or pharmaceutically acceptable salts thereof, can decrease the number and/or severity of side effects that can be attributed to a compound described herein, such as a KRAS inhibitor, or a pharmaceutically acceptable salt thereof.

Using a combination of compounds described herein can results in additive, synergistic or strongly synergistic effect. A combination of compounds described herein can result in an effect that is not antagonistic.

In some embodiments, a combination as described herein of Compound (A), including pharmaceutically acceptable salts thereof, and a KRAS inhibitor, or pharmaceutically acceptable salts thereof, can result in an additive effect. In some embodiments, a combination as described herein of Compound (A) and/or Compound (B), including pharmaceutically acceptable salts of any of the foregoing, and a KRAS inhibitor, or pharmaceutically acceptable salts thereof, can result in a synergistic effect. In some embodiments, a combination as described herein of Compound (A) and/or Compound (B), including pharmaceutically acceptable salts of any of the foregoing, and a KRAS inhibitor, or pharmaceutically acceptable salts thereof, can result in a strongly synergistic effect. In some embodiments, a combination as described herein of Compound (A) and/or Compound (B), including pharmaceutically acceptable salts of any of the foregoing, and a KRAS inhibitor, or pharmaceutically acceptable salts thereof, is not antagonistic.

As used herein, the term “antagonistic” means that the activity of the combination of compounds is less compared to the sum of the activities of the compounds in combination when the activity of each compound is determined individually (i.e., as a single compound). As used herein, the term “synergistic effect” means that the activity of the combination of compounds is greater than the sum of the individual activities of the compounds in the combination when the activity of each compound is determined individually. As used herein, the term “additive effect” means that the activity of the combination of compounds is about equal to the sum of the individual activities of the compounds in the combination when the activity of each compound is determined individually.

A potential advantage of utilizing a combination as described herein may be a reduction in the required amount(s) of the compound(s) that is effective in treating a disease condition disclosed herein compared to when each compound is administered as a monotherapy. For example, the amount of a KRAS inhibitor, or a pharmaceutically acceptable salt thereof, used in a combination described herein can be less compared to the amount of a KRAS inhibitor, or a pharmaceutically acceptable salt thereof, needed to achieve the same reduction in a disease marker (for example, tumor size) when administered as a monotherapy. Another potential advantage of utilizing a combination as described herein is that the use of two or more compounds having different mechanisms of action can create a higher barrier to the development of resistance compared to when a compound is administered as monotherapy. Additional advantages of utilizing a combination as described herein may include little to no cross resistance between the compounds of a combination described herein; different routes for elimination of the compounds of a combination described herein; and/or little to no overlapping toxicities between the compounds of a combination described herein.

### Pharmaceutical Compositions

Compound (A) and/or Compound (B), including pharmaceutically acceptable salts of any of the foregoing, can be provided in a pharmaceutical composition. Likewise, a KRAS inhibitor, including pharmaceutically acceptable salts thereof, can be provided in a pharmaceutical composition.

The term “pharmaceutical composition” refers to a mixture of one or more compounds and/or salts disclosed herein with other chemical components, such as diluents, carriers and/or excipients. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, and salicylic acid. Pharmaceutical compositions will generally be tailored to the specific intended route of administration.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. For example, stabilizers such as anti-oxidants and metal-chelating agents are excipients. In an embodiment, the pharmaceutical composition comprises an anti-oxidant and/or a metal-chelating agent. A “diluent” is a type of excipient.

In some embodiments, a KRAS inhibitor, along with pharmaceutically acceptable salts thereof, can be provided in a pharmaceutical composition that includes Compound (A) and/or Compound (B), including pharmaceutically acceptable salts of any of the foregoing. In other embodiments, a KRAS inhibitor, along with pharmaceutically acceptable salts thereof, can be administered in a pharmaceutical composition that is separate from a pharmaceutical composition that includes Compound (A) and/or Compound (B), including pharmaceutically acceptable salts of any of the foregoing.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, Compound (A) and/or Compound (B), including pharmaceutically acceptable salts of any of the foregoing, can be administered orally. In some embodiments, Compound (A) and/or Compound (B), including pharmaceutically acceptable salts of any of the foregoing, can be provided to a subject by the same route of administration as a KRAS inhibitor, along with pharmaceutically acceptable salts thereof. In other embodiments, Compound (A) and/or Compound (B), including pharmaceutically acceptable salts of any of the foregoing, can be provided to a subject by a different route of administration as a KRAS inhibitor, along with pharmaceutically acceptable salts thereof.

One may also administer the compound, salt and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical carrier may also be prepared, placed in an appropriate container, and labeled for treatment of an indicated condition.

### Uses and Methods of Treatment

As provided herein, in some embodiments, a combination of compounds that includes an effective amount of Compound (A) and/or Compound (B), including pharmaceutically acceptable salts of any of the foregoing, and an effective amount of a KRAS inhibitor, or a pharmaceutically acceptable salt of any of the foregoing, can be used to treat a disease or condition described herein, such as a cancer selected from a colorectal cancer, a pancreatic cancer and a non-small cell lung cancer.

In some cases, following cancer treatment, a subject can relapse or have reoccurrence of the cancer. As used herein, the terms “relapse” and “reoccurrence” are used in their normal sense as understood by those skilled in the art. Thus, the cancer can be a recurrent cancer.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject can be human. In some embodiments, the subject can be a child and/or an infant. In other embodiments, the subject can be an adult.

As used herein, the terms “treat,” “treating,” “treatment,” “therapeutic,” and “therapy” do not necessarily mean total cure or abolition of the disease or condition. Any alleviation of any undesired signs or symptoms of the disease or condition, to any extent can be considered treatment and/or therapy. Furthermore, treatment may include acts that may worsen the subject's overall feeling of well-being or appearance.

The term “effective amount” is used to indicate an amount of an active compound, or pharmaceutical agent, that elicits the biological or medicinal response indicated. For example, an effective amount of compound, salt or composition can be the amount needed to prevent, alleviate or ameliorate symptoms of the disease or condition, or prolong the survival of the subject being treated. This response may occur in a tissue, system, animal or human and includes alleviation of the signs or symptoms of the disease or condition being treated. Determination of an effective amount is well within the capability of those skilled in the art, in view of the disclosure provided herein. The effective amount of the compounds disclosed herein required as a dose will depend on the route of administration, the type of animal, including human, being treated and the physical characteristics of the specific animal under consideration. The dose can be tailored to achieve a desired effect, but will depend on such factors as weight, diet, concurrent medication and other factors which those skilled in the medical arts will recognize.

For example, an effective amount of a compound, or radiation, is the amount that results in: (a) the reduction, alleviation or disappearance of one or more symptoms caused by the cancer, (b) the reduction of tumor size, (c) the elimination of the tumor, and/or (d) long-term disease stabilization (growth arrest) of the tumor.

The amount of compound, salt and/or composition required for use in treatment will vary not only with the particular compound or salt selected but also with the route of administration, the nature and/or symptoms of the disease or condition being treated and the age and condition of the patient and will be ultimately at the discretion of the attendant physician or clinician. In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the dosage ranges described herein in order to effectively and aggressively treat particularly aggressive diseases or conditions.

As will be readily apparent to one skilled in the art, the useful in vivo dosage to be administered and the particular mode of administration will vary depending upon the age, weight, the severity of the affliction, the mammalian species treated, the particular compounds employed and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies. For example, useful dosages of Compounds (A), Compound (B) and/or a KRAS inhibitor, or pharmaceutically acceptable salts of the foregoing, can be determined by comparing their in vitro activity, and in vivo activity in animal models. Such comparison can be done by comparison against an established drug, such as cisplatin and/or gemcitabine.

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt or adjust administration due to toxicity or organ dysfunctions. Conversely, the attending physician would also know to adjust treatment to higher levels if the clinical response was not adequate (precluding toxicity). The magnitude of an administrated dose in the management of the disorder of interest will vary with the severity of the disease or condition to be treated and to the route of administration. The severity of the disease or condition may, for example, be evaluated, in part, by standard prognostic evaluation methods. Further, the dose and perhaps dose frequency, will also vary according to the age, body weight and response of the individual patient. A program comparable to that discussed above may be used in veterinary medicine.

Compounds, salts and compositions disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### EXAMPLES

Additional embodiments are disclosed in further detail in the following examples, which are not in any way intended to limit the scope of the claims.

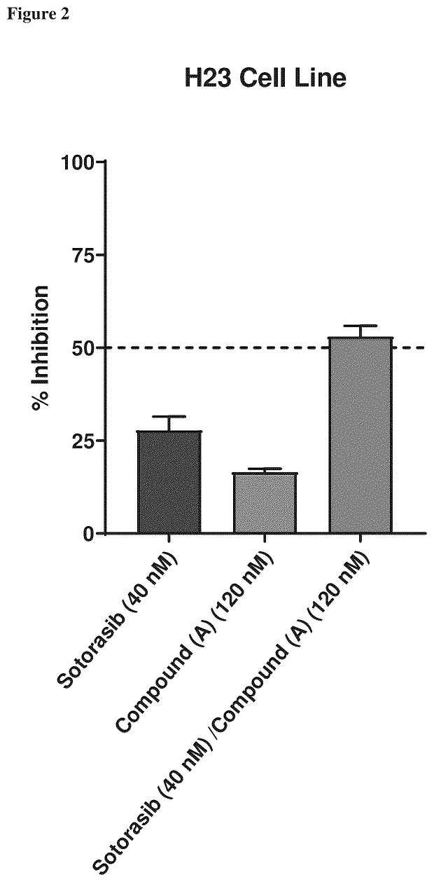

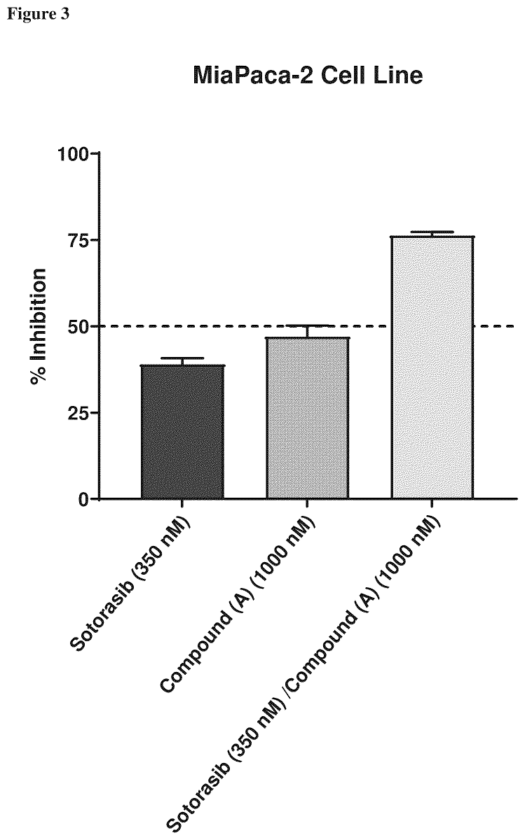

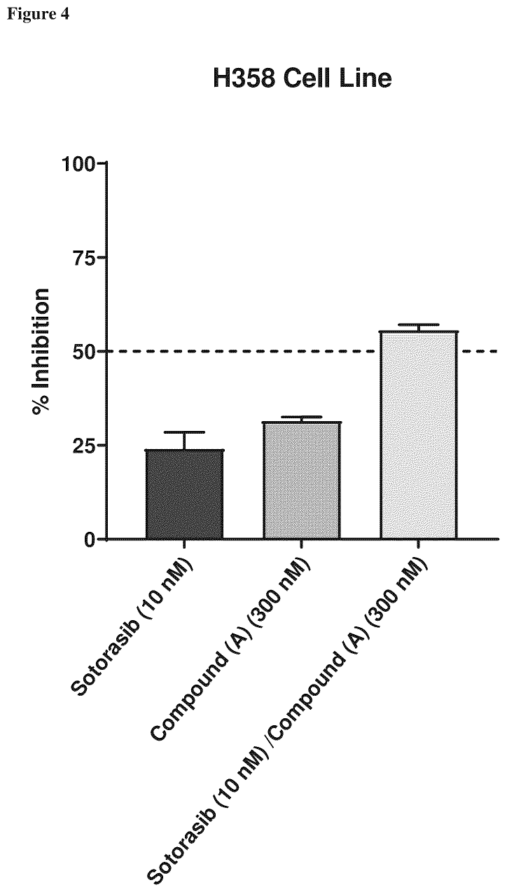

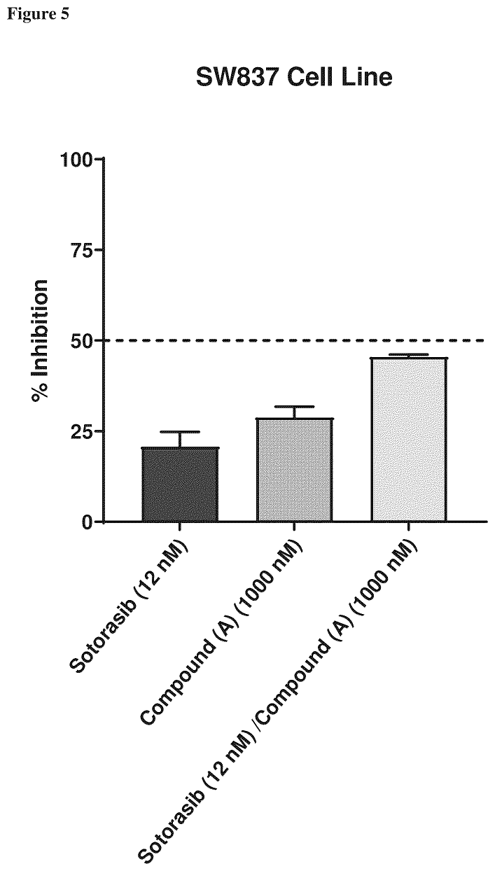

20,000 H23 cells were incubated in a 96 well plate as a triplicate with 40 nM of sotorasib or 120 nM of Compound (A) as a single agent or the combination of both for 72 h (FIG. 2). 20,000 MiaPaca-2 cells were incubated in a 96 well plate as a triplicate with 350 nM of sotorasib or 1000 nM of Compound (A) as a single agent or the combination of both for 72 h (FIG. 3). 20,000 H358 cells were incubated in a 96 well plate as a triplicate with 10 nM of sotorasib or 300 nM of Compound (A) as a single agent or the combination of both for 72 h (FIG. 4). 20,000 SW837 cells were incubated in a 96 well plate as a triplicate with 12 nM of sotorasib or 1000 nM of Compound (A) as a single agent or the combination of both for 72 h (FIG. 5). For each cell line, the cell viability was assessed using a CellTiter-Glo® (CTG) assay. Table 2 and FIGS. 2-5 provides the data and shows that the combination of Compound (A), or a pharmaceutically acceptable salt thereof, Compound (A) with a KRAS inhibitor (sotorasib) demonstrated synergistic or additive effect (CI<0.3) in all the cell lines tested.

TABLE 2

| | Cell line | | | |
|---|---|---|---|---|
| | H23 | MiaPaca-2 | H358 | SW837 |
| | % inhi- | % inhi- | % inhi- | % inhi- |
| | bition | bition | bition | bition |
| sotorasib | 27.8 | 39 | 24 | 20.7 |
| Compound (A) | 16.5 | 47 | 31.5 | 28.8 |
| sotorasib + Compound (A) | 53 | 76 | 55.6 | 45.5 |

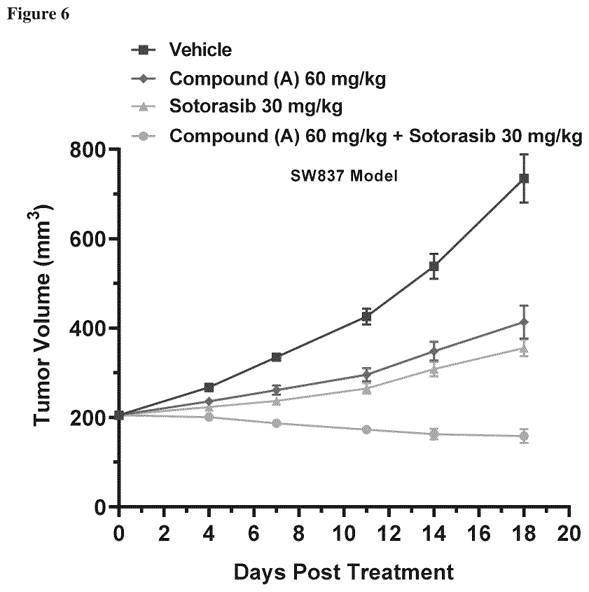

Mice were inoculated with SW837 cells subcutaneously on the right flank with the single cell suspension of 95% viable tumor cells (1×10⁷) in 100 μL L-15 Matrigel mixture (1:1 ratio) without serum for the tumor development. The treatment was started when the mean tumor size reached approximately 200 mm³ (with individual tumor range between 180-220 mm³). Animals were randomly distributed into treatment groups of 8 animals each and dosed with vehicle (top line indicated with circles) and indicated compounds at indicated dosage and frequency shown in FIG. 6 and Table 3. In FIG. 6, single agent activity was shown with Compound (A) and sotorasib at the indicated doses. In addition, the bottom line is Compound (A) 60 mg/kg p.o. qd×18+sotorasib 30 mg/kg p.o. pd×18. The combination of Compound (A) and sotorasib resulted in synergistic TGI activity and tumor regression. Tumor volumes were evaluated twice per week to calculate tumor volume over time, and mice were weighed twice per week as a surrogate for signs of toxicity. Tumor growth inhibition (TGI) was calculated using the following equation TGI=(1−(Td−T0)/(Cd−C0))×100%. Td and Cd are the mean tumor volumes of the treated and control animals, and T0 and C0 are the mean tumor volumes of the treated and control animals at the start of the experiment. The tumor regression was defined as (1−(Td/T0))×100% tumor volume (TV) decrease (Td terminal TV divided by T0 initial TV). FIG. 6 and Table 3 illustrate single agent and double agent treatment of Compound (A) at 60 mg/kg and sotorasib at 30 mg/kg. The combination of Compound (A) (60 mg/kg)+sotorasib (30 mg/kg) exhibited 109% tumor growth inhibition and 23% tumor regression at Day 18.

TABLE 3

| | TGI % | % Regression |
|---|---|---|
| Compound/Combination | (DAY 18) | (DAY 18) |
| Compound (A) 60 mg/kg | 72 | — |
| sotorasib 30 mg/kg | 80 | — |
| Compound (A) + sotorasib | 109 | 23 |

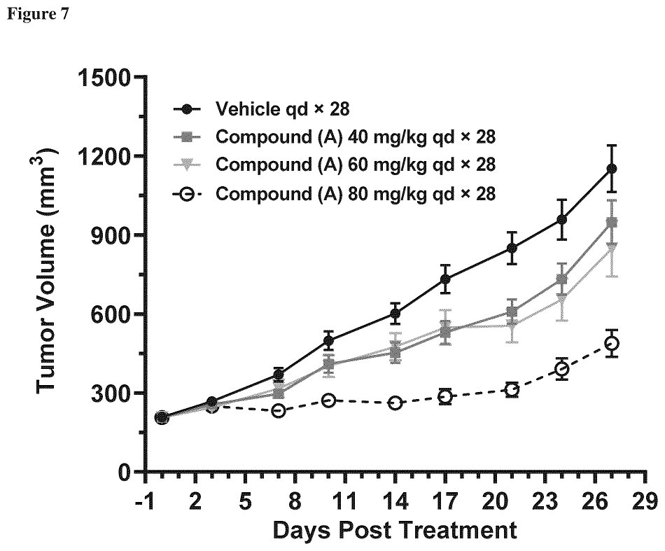

The antitumor activity of Compound (A) was assessed using the colorectal cancer LoVo xenograft model (KRAS mutant) with BALB/c nude mice. Each mouse was inoculated on the right flank subcutaneously with 5×10⁶/100 μL LoVo tumor cells for the tumor development. When the mean tumor size reached 207 mm³, animals were randomized into 4 groups (10 animals/group), and the treatments were initiated according to Table 4. FIG. 7 shows the results of this study.

TABLE 4

| | | | Animals/ | Dose | Vol | | |
|---|---|---|---|---|---|---|---|
| Model | Groups | Treatment | group | (mg/kg) | (μL/g) | Route | Regimen |
| LoVo | 1 | Vehicle | 10 | — | 10 | p.o. | qd × 28 |
| | 2 | Compound | 10 | 40 | 10 | p.o. | qd × 28 |
| | | (A) | | | | | |
| | 3 | Compound | 10 | 60 | 10 | p.o. | qd × 28 |
| | | (A) | | | | | |
| | 4 | Compound | 10 | 80 | 10 | p.o. | qd × 28 |
| | | (A) | | | | | |

Study endpoints included daily body weight, clinical observations and tumor volume. Table 5 and FIG. 7 showed that Compound (A) as a single agent produced robust inhibition of tumor growth increasing with the dose level (40 mg/kg/day, 60 mg/kg/day, and 80 mg/kg/day) with tumor growth inhibition (TGI) of 21.4%, 32.1% and 70.3%, respectively. There were no adverse clinical observations in any dose group, and there was no significant impact of treatments on mean body weights.

TABLE 5

| Compound (A) dose | TGI^{a} (%) | P value^{b} |
|---|---|---|
| 40 mg/kg/day | 21.4 | 0.098 |
| 60 mg/kg/day | 32.1 | 0.016 |
| 80 mg/kg/day | 70.3 | <0.001 |

^{a}TGI = Tumor growth inhibition, calculated as TGI = (1 − T_{d} − T₀)/(C_{d} − C₀)) × 100%;
^{b}calculated vs. Vehicle Control by LSD Test.

The antitumor activity of Compound (A) was assessed using the colorectal cancer SW1116 xenograft model (TP53 mutant; KRAS mutant) with NOD/SCID nude mice. Each mouse was inoculated on the right flank subcutaneously with 1×10⁷ (+High Concentration Matrigel)/200 μL SW1116 tumor cells for the tumor development. When the mean tumor size reached 229 mm³, animals were randomized into 4 groups (10 animals/group), and the treatments were initiated according to Table 6.

TABLE 6

| | | | Animals/ | Dose | Vol | | |
|---|---|---|---|---|---|---|---|
| Model | Groups | Treatment | group | (mg/kg) | (μL/g) | Route | Regimen |
| SW1116 | 1 | Vehicle | 10 | — | 10 | p.o. | qd × 28 |
| | 2 | Compound | 10 | 40 | 10 | p.o. | qd × 28 |
| | | (A) | | | | | |
| | 3 | Compound | 10 | 60 | 10 | p.o. | qd × 28 |
| | | (A) | | | | | |
| | 4 | Compound | 10 | 80 | 10 | p.o. | qd × 28 |
| | | (A) | | | | | |

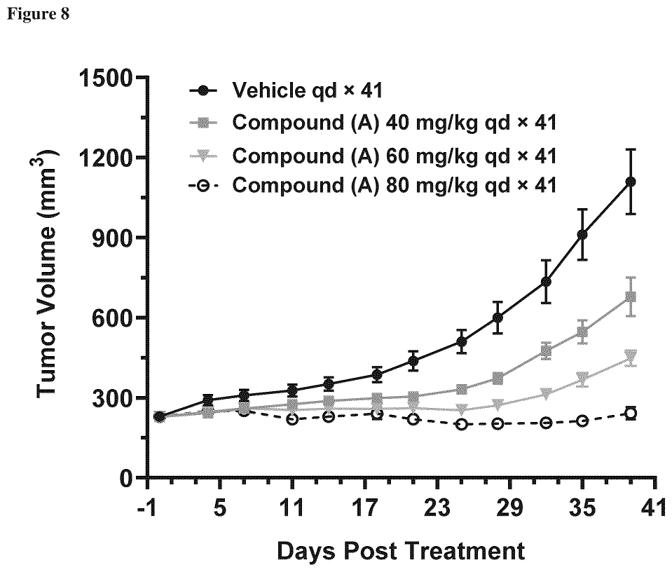

Study endpoints included daily body weight, clinical observations and tumor volume. Table 7 and FIG. 8 showed that Compound (A) as a single agent produced robust inhibition of tumor growth increasing with the dose level (40 mg/kg/day, 60 mg/kg/day, and 80 mg/kg/day) with tumor growth inhibition (TGI) of 49.0%, 75.1% and 98.5%, respectively. Treatments were generally well tolerated for the majority of study animals.

TABLE 7

| Compound (A) dose | TGI^{a} (%) | P value^{b} |
|---|---|---|
| 40 mg/kg/day | 49.0 | 0.046 |
| 60 mg/kg/day | 75.1 | 0.002 |
| 80 mg/kg/day | 98.5 | <0.001 |

^{a}TGR = Tumor growth inhibition;
^{b}calculated vs. Vehicle Control by Dunnett T3 test.

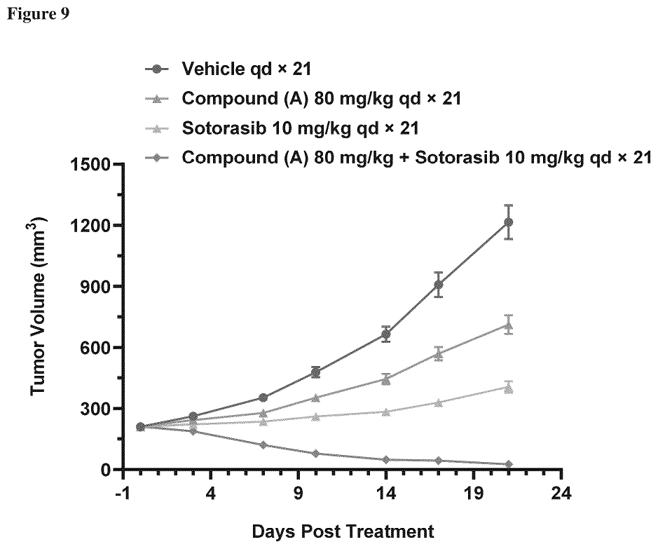

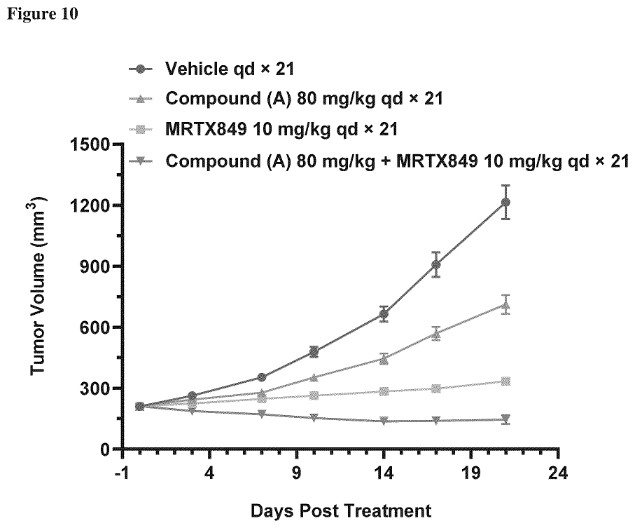

Mice were inoculated with MiaPaca-2 cells subcutaneously on the right flank with the single cell suspension of 95% viable tumor cells (1×10⁷) in 100 μL L-15 Matrigel mixture (1:1 ratio) without serum for the tumor development. The treatment was started when the mean tumor size reached approximately 200 mm³ (with individual tumor range between 180-220 mm³). Animals were randomly distributed into treatment groups of 8 animals each and dosed with vehicle (top line indicated with circles) and indicated compounds at indicated dosage and frequency shown in FIGS. 9 and 10 along with Tables 8 and 9. In FIGS. 9 and 10, single agent activity was shown with Compound (A) and sotorasib or MRTX849 at the indicated doses. In FIG. 9, the bottom line is Compound (A) 80 mg/kg p.o. qd×21+sotorasib 10 mg/kg p.o. pd×21, the second to the bottom line is sotorasib, the second from the top line is Compound (A), and the top line is vehicle. In FIG. 10, the bottom line is Compound (A) 80 mg/kg p.o. qd×21+MRTX849 10 mg/kg p.o. pd×21, the second to the bottom line is MRTX849, the second from the top line is Compound (A) and the top line is vehicle. As shown in FIGS. 9 and 10 and Tables 8 and 9, the combination of Compound (A) and sotorsaib or MRTX849 resulted in synergistic TGI activity and tumor regression.

Tumor volumes were evaluated twice per week to calculate tumor volume over time, and mice were weighed twice per week as a surrogate for signs of toxicity. Tumor growth inhibition (TGI) was calculated using the following equation TGI=(1−(Td−T0)/(Cd−C0))×100%. Td and Cd are the mean tumor volumes of the treated and control animals, and T0 and C0 are the mean tumor volumes of the treated and control animals at the start of the experiment. The tumor regression was defined as (1−(Td/T0))×100% tumor volume (TV) decrease (Td terminal TV divided by T0 initial TV). Tables 8 and 9 along with FIGS. 9 and 10 illustrate single agent and double agent treatment of Compound (A) at 80 mg/kg and sotorasib or MRTX849 at 10 mg/kg. The combination of Compound (A) (80 mg/kg)+sotorasib (10 mg/kg) exhibited 121% tumor growth inhibition and 88% tumor regression at Day 21. The combination of Compound (A) (80 mg/kg)+MRTX849 (10 mg/kg) exhibited 109% tumor growth inhibition and 31% tumor regression at Day 21.

TABLE 8

| | TGI % | % Regression |
|---|---|---|
| Compound/Combination | (DAY 21) | (DAY 21) |
| Compound (A) 80 mg/kg | 50 | — |
| sotorasib 10 mg/kg | 81 | — |
| Compound (A) + sotorasib | 121 | 88 |

TABLE 9

| | TGI % | % Regression |
|---|---|---|
| Compound/Combination | (DAY 21) | (DAY 21) |
| Compound (A) 80 mg/kg | 50 | — |
| MRTX849 10 mg/kg | 88 | — |
| Compound (A) + MRTX849 | 109 | 31 |

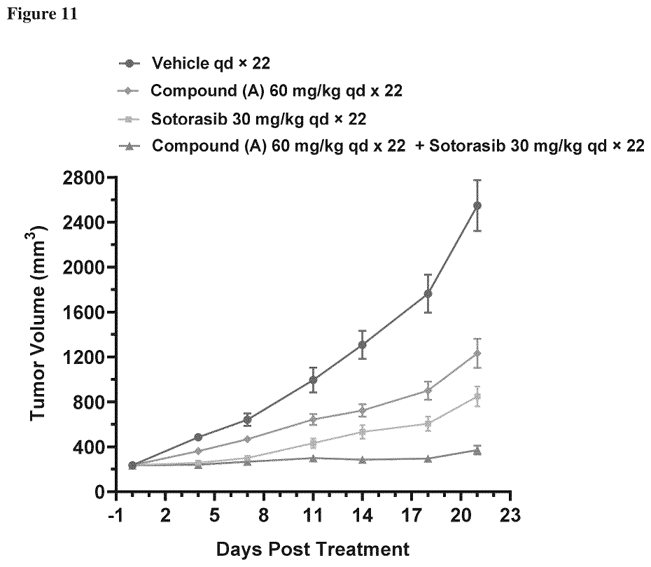

Mice were inoculated with SW1463 cells subcutaneously on the right flank with the single cell suspension of 95% viable tumor cells (1×10⁷) in 100 μL L-15 Matrigel mixture (1:1 ratio) without serum for the tumor development. The treatment was started when the mean tumor size reached approximately 200 mm³ (with individual tumor range between 180-220 mm³). Animals were randomly distributed into treatment groups of 8 animals each and dosed with vehicle (top line indicated with circles) and indicated compounds at indicated dosage and frequency shown in FIG. 11 and Table 10. In FIG. 11, single agent activity was shown with Compound (A) and sotorasib at the indicated doses, and the bottom line is Compound (A) 80 mg/kg p.o. qd×21+sotorasib 30 mg/kg p.o. pd×21. The combination of Compound (A) and sotorsaib resulted in synergistic TGI activity and tumor regression.

Tumor volumes were evaluated twice per week to calculate tumor volume over time, and mice were weighed twice per week as a surrogate for signs of toxicity. Tumor growth inhibition (TGI) was calculated using the following equation TGI=(1−(Td−T0)/(Cd−C0))×100%. Td and Cd are the mean tumor volumes of the treated and control animals, and T0 and C0 are the mean tumor volumes of the treated and control animals at the start of the experiment. The tumor regression was defined as (1−(Td/T0))×100% tumor volume (TV) decrease (Td terminal TV divided by T0 initial TV). FIG. 11 and Table 10 illustrate single agent and double agent treatment of Compound (A) at 80 mg/kg and sotorasib at 30 mg/kg. The combination of Compound (A) (80 mg/kg)+sotorasib (30 mg/kg) exhibited 94% tumor growth inhibition at Day 21.

TABLE 10

| | TGI % | % Regression |
|---|---|---|
| Compound/Combination | (DAY 21) | (DAY 21) |
| Compound (A) 80 mg/kg | 57 | — |
| sotorasib 30 mg/kg | 73 | — |
| Compound (A) + sotorasib | 94 | — |

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the present disclosure.

What is claimed is:

1. Use of an effective amount of Compound (A) for treating a cancer in a subject, wherein:
Compound (A) is 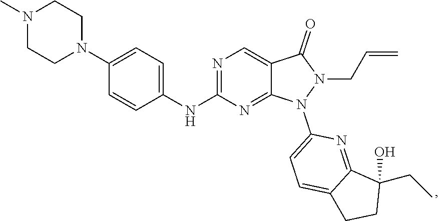 [SMILES: C=CCn1c(=O)c2cnc(Nc3ccc(N4CCN(C)CC4)cc3)nc2n1-c1ccc2c(n1)[C@@](O)(CC)CC2]
or a pharmaceutically acceptable salt thereof;
wherein the cancer is selected from a colorectal cancer, a pancreatic cancer and a non-small cell lung cancer; and
wherein the subject has a mutation selected from the group consisting of a TP53 and a KRAS mutation.

2. Use of an effective amount of Compound (B) for treating a cancer in a subject, wherein:
Compound (B) is AZD-1775, or a pharmaceutically acceptable salt thereof;
wherein the cancer is selected from a colorectal cancer, a pancreatic cancer and a non-small cell lung cancer; and
wherein the subject has a mutation selected from the group consisting of a TP53 and a KRAS mutation.

3. The use of claim 1 or 2, wherein the subject is further administered an effective amount of a KRAS inhibitor selected from the group consisting of sotorasib, adagrasib, JDQ443, MRTX-1257, MRTX1133, ARS-1620, ARS-853, ARS-107, BAY-293, BI-3406, BI-2852, BMS-214662, MRTX849, MRTX849-VHL (LC2), PROTAC K-Ras Degrader-1 (Compound 518, CAS No. 2378258-52-5), Lonafarnib (SCH66336), RMC-0331, GDC-6036, LY3537982, D-1553, ARS-3248 (JNJ74699157), BI-1701963, and AU-8653 (AU-BEI-8653).

4. The use of claim 3, wherein the KRAS inhibitor is sotorasib.

5. The use of claim 3, wherein the KRAS inhibitor is adagrasib

6. The use of claim 3, wherein the KRAS inhibitor is MRTX849.

7. The use of any one of claims 1-6, wherein the mutation is a KRAS mutation.

8. The use of any one of claims 1-6, wherein the mutation is a TP53 mutation.

9. The use of any one of claims 1-6, wherein the mutation is a TP53 and KRAS mutation.

10. The use of any one of claims 1-9, wherein the cancer is a colorectal cancer.

11. The use of any one of claims 1-9, wherein the cancer is a pancreatic cancer.

12. The use of any one of claims 1-9, wherein the cancer is a non-small cell lung cancer.
